(19) United States
(12) Patent Application Publication
(10) Pub. No.: US 2022/0356440 A1
(43) Pub. Date: Nov. 10, 2022

(54) METHOD OF PRODUCING BOTULINUM TOXIN

(71) Applicants: GALDERMA HOLDING SA, Zug (CH); IPSEN BIOPHARM LIMITED, Wrexham (GB)

(72) Inventors: Ulf STAHL, Uppsala (US); Peter FRANK, Uppsala (SE); Anders JARSTAD, Uppsala (SE); Andrew PICKETT, Uppsala (SE)

(21) Appl. No.: 17/757,719

(22) PCT Filed: Dec. 19, 2020
(86) PCT No.: PCT/IB2020/062252

§ 371 Date: Jun. 20, 2022

Related U.S. Application Data
(60) Provisional application No. 62/951,549, filed on Dec. 20, 2019.

(51) Int. Cl. C12N 1/20

(52) U.S. Cl. CPC C12N 1/205; C12R 2001/145

(57) ABSTRACT

The present disclosure relates generally to the field of producing botulinum toxin. More specifically, the present disclosure relates to a method for producing botulinum toxin in a culture medium free or substantially free of animal product. The present disclosure also relates to the culture medium for producing botulinum toxin that is free or substantially free of animal product.

### CROSS-REFERENCE TO RELATED APPLICATIONS

This application claims priority under 35 U.S.C. § 119(e) to U.S. Provisional Application No. 62/951,549, filed Dec. 20, 2019, which is hereby incorporated by reference in its entirety.

### FIELD

The present disclosure relates generally to the field of producing botulinum toxin. More specifically, the present disclosure relates to a method for producing botulinum toxin in a culture medium free or substantially free of animal product. The present disclosure also relates to the culture medium for producing botulinum toxin that is free or substantially free of animal product.

### BACKGROUND

The following description of the background of the present technology is provided simply as an aid in understanding the present technology and is not admitted to describe or constitute prior art to the present technology.

Seven generally immunologically distinct botulinum neurotoxins have been characterized—botulinum neurotoxin serotypes A, B, C, D, E, F, and G—each of which is distinguished by neutralization with type-specific antibodies. As one example, BOTOX® is the trademark of a botulinum toxin type A purified neurotoxin complex available commercially from Allergan, Inc., of Irvine, Calif. Botox is a popular injection-based cosmetic treatment that temporarily reduces the appearance of fine lines and wrinkles. One unit (U) of botulinum toxin is defined as the LD₅₀ upon intraperitoneal injection into female Swiss Webster mice weighing 18-20 grams each. In other words, one unit of botulinum toxin is the amount of botulinum toxin that kills 50% of a group of female Swiss Webster mice. Seven generally immunologically distinct botulinum neurotoxins have been characterized, these being respectively botulinum neurotoxin serotypes A, B, C, D, E, F, and G, each of which is distinguished by neutralization with type-specific antibodies. The different serotypes of botulinum toxin vary in the animal species that they affect and in the severity and duration of the paralysis they evoke. For example, it has been determined that botulinum toxin type A is 500 times more potent, as measured by the rate of paralysis produced in the rat, than is botulinum toxin type B. Additionally, botulinum toxin type B has been determined to be non-toxic in primates at a dose of 480 U/kg which is about 12 times the primate LD5o for botulinum toxin type A. It is known that botulinum toxins can also be utilized to treat a variety of disorders. Examples include U.S. Pat. No. 5,714,468 (migraine) issued Feb. 3, 1998; Published U.S. Patent Application No. 2005019132 (headache), Ser. No. 11/039,506, filed Jan. 18, 2005; Published U.S. Patent Application No. 20050191320 (medication overuse headache), Ser. No. 10/789,180, filed Feb. 26, 2004; and U.S. Pat. No. 7,811,587 (neuropsychiatric disorders), issued Oct. 12, 2010; all incorporated entirely by reference.

Botulinum toxin is conventionally obtained through a culturing and fermentation process which uses one or more animal derived product (such as a meat broth culture medium, and a blood fraction or blood derivative excipient). Administration to a patient of a pharmaceutical composition wherein the active ingredient biologic is obtained through a process which makes use of animal derived products can subject the patient to a potential risk of receiving various pathogens or infectious agents. For example, prions may be present in a pharmaceutical composition. A prion is a proteinaceous infectious particle which is hypothesized to arise as an abnormal conformational isoform from the same nucleic acid sequence which makes the normal protein. It has been further hypothesized that infectivity resides in a “recruitment reaction” of the normal isoform protein to the prion protein isoform at a post translational level. Apparently the normal endogenous cellular protein is induced to misfold into a pathogenic prion conformation.

There is a need for the development of a process for producing botulinum toxin with a culture medium free or substantially free of animal derived product that minimizes the risks and problems associated with undesirable contaminants from animals.

### SUMMARY

Provided herein are methods for production of a botulinum toxin, comprising the steps of (a) providing a working cell bank (WCB) comprising a Clostridum botulinum bacterium; (b) adding the working cell bank to a first container containing a vegetable toxin production medium (VTPM), and culturing the Clostridium botulinum bacterium in the VTPM under conditions which permit growth of the Clostridium botulinum to produce a pre-culture; (c) adding the pre-culture to a second container containing VTPM, and culturing the Clostridium botulinum bacterium under conditions that allow production of a botulinum toxin, and; (d) recovering the botulinum toxin; wherein the VTPM is substantially free or free of an animal derived product and comprises a plant-derived protein.

In some embodiments, the botulinum toxin is botulinum neurotoxin type A (BoNT/A).

In some embodiments, the container used in the steps (b) and (c) is a fermentation bag.

In some embodiments, the conditions in steps (b) and (c) comprise an anaerobic environment. In some embodiments, the anaerobic environment has a dissolved oxygen (DO) concentration of <2%. In some embodiments, the anaerobic environment has a dissolved oxygen (DO) concentration of <1%. In some embodiments, the anaerobic environment has a dissolved oxygen (DO) concentration of <0.5%.

In some embodiments, the condition in step (b) comprises a temperature of between about 35° C. and about 39° C., or between about 36° C. and about 38° C. (or ranges in between). In some embodiments, the condition in step (b) comprises a temperature of about 35.0° C., about 35.5° C., about 36.0° C., about 36.5° C., about 37.0° C., about 37.5° C., about 38.0° C., about 38.5° C., or about 39.0° C. In some embodiments, the condition in step (b) comprises a temperature of about 37±1° C. In some embodiments, the condition in step (b) comprises a temperature of about 37±0.5° C. In some embodiments, the condition in step (b) comprises a temperature of about 37±0.2° C.

In some embodiments, the condition in step (c) comprises a temperature of between about 30° C. and about 37° C., between about 31° C. and about 36° C., between about 32° C. and about 35° C., or between about 32° C. and about 34° C. (or ranges in between). In some embodiments, the condition in step (c) comprises a temperature of about 33±1° C. In some embodiments, the condition in step (c) comprises a temperature of about 33±0.5° C. In some embodiments, the condition in step (c) comprises a temperature of about 33±0.2° C.

In some embodiments, the volume ratio of the WCB to the VTPM in step (b) is no greater than about 2.0%, no greater than about 1.9%, no greater than about 1.8%, no greater than about 1.7%, no greater than about 1.6%, no greater than about 1.5%, no greater than about 1.4%, no greater than about 1.3%, no greater than about 1.2%, no greater than about 1.1%, no greater than about 1.0%, no greater than about 0.9%, no greater than about 0.8%, no greater than about 0.7%, no greater than about 0.6%, no greater than about 0.5%, no greater than about 0.4%, no greater than about 0.3%, no greater than about 0.2%, no greater than about 0.1%, no greater than about 0.09%, no greater than about 0.08%, no greater than about 0.07%, no greater than about 0.06%, no greater than about 0.05%, no greater than about 0.04%, no greater than about 0.03%, no greater than about 0.02%, or no greater than about 0.01% (or ranges in between).

In some embodiments, the volume ratio of the WCB to the VTPM in step (b) is about 0.01%, about 0.02%, about 0.03%, about 0.04%, about 0.05%, about 0.06%, about 0.07%, about 0.08%, about 0.09%, about 0.10%, about 0.15%, about 0.20%, about 0.25%, about 0.30%, about 0.35%, about 0.40%, about 0.45%, about 0.50%, about 0.55%, about 0.60%, about 0.65%, about 0.70%, about 0.75%, about 0.80%, about 0.85%, about 0.90%, about 0.95%, about 1.0%, about 1.1%, about 1.2%, about 1.3%, about 1.4%, about 1.5%, about 1.6%, about 1.7%, about 1.8%, about 1.9%, or about 2.0%.

In some embodiments, the volume ratio of the pre-culture to the VTPM in step (c) is between about 1:2 and about 1:50, between about 1:3 and about 1:45, between about 1:4 and about 1:40, between about 1:5 and about 1:35, between about 1:6 and about 1:30, between about 1:7 and about 1:25, between about 1:8 and about 1:20, or between about 1:8 and about 1:10 (or ranges in between). In some embodiments, the volume ratio of the pre-culture to the VTPM in step (c) is about 1:2, about 1:3, about 1:4, about 1:5, about 1:6, about 1:7, about 1:8, about 1:9, about 1:10, about 1:15, about 1:20, about 1:25, about 1:30, about 1:35, about 1:40, about 1:45, or about 1:50.

In some embodiments, the step (b) is conducted until OD₆₀₀ reaches the range of about 0.1 to about 1.0, about 0.1 to about 0.05, or about 0.2 to about 0.4. In some embodiments, the step (b) is conducted until OD₆₀₀ reaches about 0.1, about 0.2, about 0.3, about 0.4, about 0.5, about 0.6, about 0.7, about 0.8, about 0.9, or about 1.0.

In some embodiments, the step (b) is conducted for between about 10 and about 30 hours, between about 15 and about 25, hours or between about 17 and about 21 hours (or ranges in between). In some embodiments, the step (b) is conducted for about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, or about 30 hours. In some embodiments, the step (b) is conducted for about 19±2 hours. In some embodiments, the step (b) is conducted for about 19±1 hours. In some embodiments, the step (b) is conducted for about 19±0.5 hours. In some embodiments, the step (b) is conducted for about 19±0.2 hours. In some embodiments, the step (b) is conducted for about 19 hours.

In some embodiments, the step (c) is carried out for between about 60 hours and about 80 hours, between about 65 hours and about 75 hours, or between about 67 hours and about 71 hours (or ranges in between). In some embodiments, the fermentation process continues for about 60 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, or about 80 hours. In some embodiments, the fermentation process continues for about 69±2 hours, about 69±1 hours, about 69±0.5 hours, or about 69±0.2 hours. In an embodiment, the step (c) is conducted for about 69 hours.

In some embodiments, microbiological purity for other microorganisms than C. botulinum is tested in the pre-culture after step (b) and before step (c).

In some embodiments, microbiological purity for other microorganisms than C. botulinum is tested in the culture after step (c) and before step (d).

In some embodiments, the plant-derived protein is a wheat peptone. In some embodiments, the wheat peptone concentration in the VTPM is between about 10 grams per liter and about 30 grams per liter, for example, about 20 grams per liter. In some embodiments, the wheat peptone concentration in the VTPM is about 20 grams per liter. In some embodiments, the VTPM comprises wheat peptone, yeast extract, D-(+)-Glucose, L-Cysteine hydrochloride monohydrate, Medical antifoam C emulsion.

In some embodiments, the VTPM comprises wheat peptone, yeast extract, D-(+)-Glucose, L-Cysteine hydrochloride monohydrate, Medical antifoam C emulsion, Distilled water, NaOH and HCl. In a specific embodiment, the VTPM comprises about 20 grams per liter of wheat peptone, about 20 grams per liter of yeast extract, about 5 grams per liter of D-(+)-glucose; about 0.20 grams per liter of L-cysteine hydrochloride monohydrate and about 0.24 grams per liter of medical antifoam c emulsion. In a specific embodiment, the pH of the VTPM is between about 6.7 and about 7.2.

In another aspect, provided herein are compositions comprising a Clostridium botulinum and a culture medium for producing a botulinum toxin, wherein the medium is free or substantially free of an animal derived product, and comprises one or more plant-derived proteins. In some embodiments, the one or more plant-derived proteins is a wheat peptone, broadbean peptone, potato peptone, pea peptone, rice peptone, or soybean peptone, or combinations thereof. In some embodiments, the plant-derived protein is a wheat peptone.

In some embodiments, the wheat peptone concentration in the VTPM is about 20 grams per liter. In some embodiments, the VTPM comprises wheat peptone, yeast extract, D-(+)-Glucose, L-Cysteine hydrochloride monohydrate, Medical antifoam C emulsion.

In some embodiments, the VTPM comprises wheat peptone, yeast extract, D-(+)-Glucose, L-Cysteine hydrochloride monohydrate, Medical antifoam C emulsion, Distilled water, NaOH and HCl. In a specific embodiment, the VTPM comprises about 20 grams per liter of wheat peptone, about 20 grams per liter of yeast extract, about 5 grams per liter of D-(+)-glucose; about 0.20 grams per liter of L-cysteine hydrochloride monohydrate and about 0.24 grams per liter of medical antifoam c emulsion.

### BRIEF DESCRIPTION OF THE DRAWINGS

FIG. 1 illustrates the fermentation process. 400 μl of working cell bank (WCB) was added to 500 mL growth media in 2 L fermentation bag. The bag was flushed with filtered nitrogen prior to inoculation. Fermentation was carried out until OD₆₀₀ reached 0.2-0.4 at 37±° C. Next, 4500 mL vegetable toxin production medium (VTPM) was added to 5 L cultivation bag containing 500 mL pre-culture. The bag was flushed with filtered nitrogen prior to inoculation. Fermentation was conducted for 69±2 h at 33±1° C.

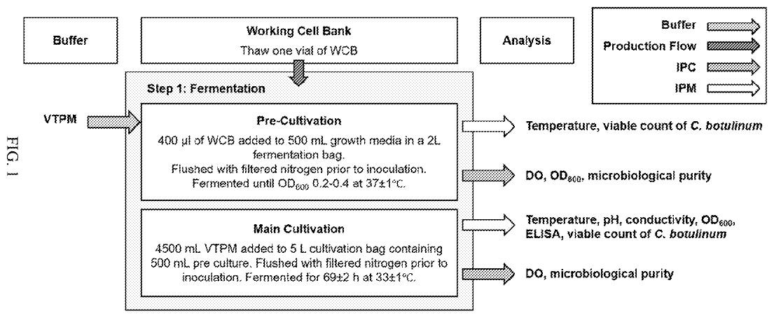

FIG. 2 shows the curve of optical density at 600 nm for the main cultivation. The curve is based on samples withdrawn an analyzed from a few fermentations performed as stated in Example 2 below.

FIG. 3 shows the pH-curve for the main cultivation. The curve is based on samples withdrawn and analyzed from a few fermentations performed according to Example 2 below.

FIG. 4 shows a Western blot analysis of BoNT/A heavy chain variants in a main cultivation performed according to Example 2. Samples were withdrawn at different time points in the fermentation and are run together with reference samples showing either only band 2 or both band 1 and band 2.

FIG. 5 shows a table with cut-out figures from Western blot analysis of BoNT/A heavy chain variants in samples at harvest of main cultivations at different temperatures. The tables also provide BoNT/A concentrations for the same samples, as determined by ELISA.

FIG. 6 shows toxin content obtained at harvest at 69 h of main cultures grown in VTPM based on wheat peptone or VTPM based on soy bean peptone.

FIG. 7 shows toxin concentration obtained at harvest at 69 h of main cultures grown in VTPM based on peptone from potato, broadbean, or wheat.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. It is to be understood that this present technology is not limited to particular methods, reagents, compounds compositions or biological systems, which can, of course, vary. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Unless the context indicates otherwise, it is specifically intended that the various features of the technology described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All patents, patent applications, provisional applications, and publications referred to or cited herein are incorporated by reference in their entirety, including all figures and tables, to the extent they are not inconsistent with the explicit teachings of this specification.

Definitions

As used herein, the singular forms “a,” “an,” and “the” designate both the singular and the plural, unless expressly stated to designate the singular only.

It is to be understood, although not always explicitly stated, that all numerical designations are preceded by the term “about” or “approximately.” The term “about” or “approximately” means that the number comprehended is not limited to the exact number set forth herein, and is intended to refer to numbers substantially around the recited number while not departing from the scope of the invention. As used herein, “about” “or “approximately” will be understood by persons of ordinary skill in the art and will vary to some extent on the context in which it is used. If there are uses of the term which are not clear to persons of ordinary skill in the art given the context in which it is used, “about” or “approximately” will mean the recited term and up to plus or minus 15%, 10%, 5%, 1%, or 0.1% of the particular term (e.g., “about 10” should be understood as 10 and a range of up to 8.5-11.5).

Also as used herein, “and/or” refers to and encompasses any and all possible combinations of one or more of the associated listed items, as well as the lack of combinations when interpreted in the alternative (“or”).

As used herein, “animal product free,” “essentially animal product free,” or “substantially animal product free” encompass, respectively, “animal protein free,” “essentially animal protein free,” or “substantially animal protein free,” and means the absence, essential absence, or substantial absence of blood derived, blood pooled and other animal derived products or compounds. “Animal” means a mammal (such as a human), bird, reptile, fish, insect, spider or other animal species. “Animal” excludes microorganisms, such as bacteria. Thus, an animal product free medium or process or a substantially animal product free medium or process within the scope of the present disclosure can include a botulinum toxin or a Clostridial botulinum bacterium. For example, an animal product free process or a substantially animal product free process means a process which is either substantially free or essentially free or entirely free of animal derived proteins, such as immunoglobulins, meat digest, meat by products and milk or dairy products or digests. Thus, an example of an animal product free process is a process (such as a bacterial culturing or bacterial fermentation process) which excludes meat and dairy products or meat or dairy by products.

As used herein, “botulinum toxin” means a neurotoxin produced by Clostridium botulinum, as well as a botulinum toxin (or the light chain or the heavy chain thereof) made recombinantly by a non-Clostridial species. The phrase “botulinum toxin”, as used herein, encompasses the botulinum toxin serotypes A, B, C, D, E, F and G. Botulinum toxin, as used herein, also encompasses both a botulinum toxin complex (i.e. the 300, 600 and 900 kDa complexes) as well as the purified botulinum toxin (i.e. about 150 kDa). “Purified botulinum toxin” is defined as a botulinum toxin that is isolated, or substantially isolated, from other proteins, including proteins that form a botulinum toxin complex. A purified botulinum toxin may be greater than 95% pure, and preferably is greater than 99% pure. The botulinum C2 and C3 cytotoxins, not being neurotoxins, are excluded from the scope of the present disclosure. As used herein, “botulinum toxin” also encompasses “modified botulinum toxin.”

“Modified botulinum toxin” means a botulinum toxin that has had at least one of its amino acids deleted, modified, or replaced, as compared to a native botulinum toxin. Additionally, the modified botulinum toxin can be a recombinantly produced neurotoxin, or a derivative or fragment of a recombinantly made neurotoxin. A modified botulinum toxin retains at least one biological activity of the native botulinum toxin, such as, the ability to bind to a botulinum toxin receptor, or the ability to inhibit neurotransmitter release from a neuron. One example of a modified botulinum toxin is a botulinum toxin that has a light chain from one botulinum toxin serotype (such as serotype A), and a heavy chain from a different botulinum toxin serotype (such as serotype B). Another example of a modified botulinum toxin is a botulinum toxin coupled to a neurotransmitter, such as substance P.

“Medium” or “fermentation medium,” as used herein, means any medium for cultivating bacteria, either for growth in order to produce a seed culture to be used for inoculation of the production medium, or the production medium in which the bacteria grow and produce their toxin. An example of fermentation medium according to the present disclosure is a vegetable toxin producing medium (VTPM).

As used herein, “Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species. Clostridia toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, 7B, FE and /F), animals (BoNT/C and /D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently five BoNT/A subtypes, BoNT/A1, BoNT/A2, BoNT/A3, BoNT/A4 and BoNT/A5, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other species of Clostridia, C. baratii and C. butyricum, also produce toxins, BaNT and BuNT respectively, which are similar to BoNT/F and BoNT/E, respectively.

Clostridial toxins are released by Clostridial bacterium as complexes comprising the approximately 150-kDa Clostridial toxin along with associated non-toxin proteins (NAPs). Identified NAPs include proteins possessing hemaglutination activity, such, e.g., a hemagglutinin of approximately 17-kDa (HA-17), a hemagglutinin of approximately 33-kDa (HA-33) and a hemagglutinin of approximately 70-kDa (HA-70); as well as non-toxic non-hemagglutinin (NTNH), a protein of approximately 130-kDa, see, e.g., Eric A. Johnson and Marite Bradshaw, Clostridial botulinum and its Neurotoxins: A Metabolic and Cellular Perspective, 39 Toxicon 1703-1722 (2001); and Stephanie Raffestin et al., Organization and Regulation of the Neurotoxin Genes in Clostridium botulinum and Clostridium tetani, 10 Anaerobe 93-100 (2004). Thus, the botulinum toxin type A complex can be produced by Clostridial bacterium as 900-kDa, 500-kDa and 300-kDa forms. Botulinum toxin types B and C are apparently produced as only a 500-kDa complex. Botulinum toxin type D is produced as both 300-kDa and 500-kDa complexes. Finally, botulinum toxin types E and Fare produced as only approximately 300-kDa complexes. The differences in molecular weight for the complexes are due to differing ratios of NAPs. The toxin complex is important for the intoxication process because it provides protection from adverse environ mental conditions, resistance to protease digestion, and appears to facilitate internalization and activation of the toxin.

Clostridial toxins are each translated as a single chain polypeptide that is subsequently cleaved by proteolytic Scission within a disulfide loop by a naturally-occurring protease. This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains. The naturally-occurring protease used to convert the single chain molecule into the di-chain is currently not known. In some serotypes, such as, e.g., BoNT/A, the naturally-occurring protease is produced endogenously by the bacteria serotype and cleavage occurs within the cell before the toxin is release into the environment. However, in other serotypes, such as, e.g., BoNT/E, the bacterial Strain appears not to produce an endogenous protease capable of converting the single chain form of the toxin into the di-chain form. In these situations, the toxin is released from the cell as a single-chain toxin which is subsequently converted into the di-chain form by a naturally-occurring protease found in the environment.

“Free,” or “entirely free,” as used herein, means that within the detection range of the instrument or process being used, the substance cannot be detected, or its presence cannot be confirmed.

“Essentially free,” as used herein, means that only trace amounts of the substance can be detected. In the present disclosure, “essentially free” means that the substance is at a level of less than 0.1%, preferably less than 0.01%, and most preferably, less than 0.001% by weight of the entire composition.

“Substantially free,” as used herein, means that the substance is at a level of less than 5%, preferably less than 2%, and most preferably, less than 1% by weight of the entire composition.

As used herein, “medium” or “fermentation medium” means any medium for cultivating bacteria either for growth in order to produce a seed culture to be used for inoculation of the production medium, or the production medium in which the bacteria grow and produce their toxin.

As used herein, “working cell back” or “WCB” means a population of essentially homologous cells originating from a single master cell bank (MCB). WCB are commonly required during therapeutic development and manufacturing. WCB are produced from a single vial of the MCB that have been grown for several passages and cryopreserved. In other words, cells for a WCB are expanded from an MCB. Indeed, when a cell line is to be used over many manufacturing cycles, a two-tiered cell banking system consisting of a master cell bank (MCB) and a working cell bank (WCB) is widely recommended.

As used herein, “vegetable toxin production medium” or “VTPM” means a cell culture medium that contains a component or components derived from one or more vegetables (e.g., wheat, soy, broadbean, potato, pea, etc.). The component or components derived from one or more vegetables may include, but are not limited to, a vegetable digest, a peptone, or an extract.

As used herein, “peptone” means hydrolyzed proteinaceous material formed by enzymatic or acid digestion.

As used herein, “vegetable extract” means aqueous extracts of any vegetable, containing amino acids and low molecular weight peptides, carbohydrates, vitamins and other growth factors.

As used herein, “plant peptone” means proteinaceous material, derived from plants, which has been hydrolyzed by use of microbial or vegetable enzymes, or by acid hydrolysis. The protein substrate for forming peptones may be any proteinaceous material derived from vegetables, or protein concentrate isolated from flour of e.g. rice, wheat, or soy. The term “yeast peptone” means proteinaceous material derived from yeast cells, which has been hydrolyzed by autolysis, or by use of microbial or vegetable enzymes, or by acid hydrolysis. The plant peptone in this disclosure refers to partial digestion product of plant-derived protein, which is in the form of a mixture that includes not only single molecule of amino acid, but also peptides consisting of few or several tens of amino acids and intact protein molecules. Preferably, plant peptone in this disclosure is soy peptone, wheat peptone, broadbean peptone, potato peptone, pea peptone, Papaic soy peptone or lupin peptone, most preferably pea peptone and wheat peptone.

As used herein, “OD₆₀₀” means the optical density measured at a wavelength of 600 nm. A person of ordinary skill in the art will recognize that OD₆₀₀ measurement is a common method for estimating the concentration of cells (including bacteria) in a liquid. Methods for determining OD₆₀₀ are described, for example, by S.A. Janke, et al., Microbiological Turbidity Using Standard Photometers, 6 BIOSPEKTRUM 501-02 (1999); K. Harnack, et al. Turbidity Measurements (OD₆₀₀) with Absorption Spectrometers, 6 BIOSPEKTRUM 503-04 (1999).

Vegetable Toxin Producing Medium (VTPM)

Vegetable extracts can be used in media for growth of pathogenic bacteria and production of their toxins. Vegetable extract are aqueous extracts of plants containing amino acids and low molecular weight peptides, relatively high concentrations of carbohydrates, vitamins and other growth factors. In accordance with the present disclosure, plant-derived proteins, such as peptones derived from plants, including potato, wheat, rice, a mixture of wheat and rice, cotton, or pea, can substitute animal derived product to support the growth of Clostridium botulinum bacterium. Peptones that may be used for the purposes of the disclosed VTPM can include, but are not limited to wheat peptone CAS #94350-06-8, wheat peptone E1, wheat peptone E260, pea peptone CAS #100209-45-8, pea peptone A482, pea peptone A2501, potato peptone CAS #100209-45-8, potato peptone E210, potato peptone L8, potato peptone A2401, rice peptone 19560, cotton peptone 200, soy peptone CAS #91079-46-8, soy peptone A3SC, soy peptone A2SC, and other plant or vegetable peptones.

In some embodiments, the peptone is a wheat peptone. In specific embodiments, the concentration of the wheat peptone in the fermentation medium is between 5-50 g/L, preferably between 10-40 g/L, preferably between 15-30 g/L, preferably between 15-25 g/L, and more preferably about 20 g/L of the fermentation medium. In some embodiments, the concentration of the wheat peptone in the fermentation medium is about 5 g/L, about 10 g/L, about 15 g/L, about 20 g/L, about 25 g/L, about 30 g/L, about 35 g/L, about 40 g/L, about 45 g/L, or about 50 g/L.

In accordance with the present disclosure, the fermentation medium comprises a yeast extract. Yeast extracts are generally obtained by salt-free autolysis of primary yeast and subsequent extensive purification, which renders the yeast extract free from undesired components such as spores and DNA.

In accordance with the present disclosure, the fermentation medium further comprises yeast extract. In some embodiments, the concentration of the yeast extract in the fermentation medium is between 5-50 g/L, preferably between 10-40 g/L, preferably between 15-30 g/L, preferably between 15-25 g/L, and more preferably about 20 g/L of the fermentation medium. In some embodiments, the concentration of the yeast extract in the fermentation medium is about 5 g/L, about 10 g/L, about 15 g/L, about 20 g/L, about 25 g/L, about 30 g/L, about 35 g/L, about 40 g/L, about 45 g/L, or about 50 g/L.

Various carbon sources have been used to grow C. botulinum including glucose and glycerol. The addition of a separate carbon source is not absolutely necessary if a carbon-containing nitrogen source is used (C. botulinum can assimilate carbon from amino acids), but growth rates are much higher during fermentation if an additional carbon source is present.

In accordance with the present disclosure, the fermentation medium comprises D-(+)-Glucose. In some embodiments, the concentration of the D-(+)-Glucose in the fermentation medium is between 0.5-20 g/L, preferably between 1.0-10 g/L, preferably between 2.5-7.5 g/L, preferably between 3.5-6.5 g/L, and more preferably about 5 g/L of the fermentation medium. In some embodiments, the concentration of the D-(+)-Glucose in the fermentation medium is about 0.5 g/L, about 0.6 g/L, about 0.7 g/L, about 0.8 g/L, about 0.9 g/L, about 1.0 g/L, about 1.5 g/L, about 2.0 g/L, about 2.5 g/L, about 3.0 g/L, about 3.5 g/L, about 4.0 g/L, about 4.5 g/L, about 5.0 g/L, about 5.5 g/L, about 6.0 g/L, about 6.5 g/L, about 7.0 g/L, about 7.5 g/L, about 8.0 g/L, about 8.5 g/L, about 9.0 g/L, about 9.5 g/L, about 10.0 g/L, about 11.0 g/L, about 12.0 g/L, about 13.0 g/L, about 14.0 g/L, about 15.0 g/L, about 16.0 g/L, about 17.0 g/L, about 18.0 g/L, about 19.0 g/L, or about 20.0 g/L.

In accordance with the present disclosure, the fermentation medium also comprises L-Cysteine hydrochloride monohydrate. In some embodiments, the concentration of the L-Cysteine hydrochloride monohydrate in the fermentation medium is between 0.05-5 g/L, preferably between 0.1 -5 g/L, preferably between 0.1-2.5 g/L, preferably between 0.15-1.5 g/L, and more preferably about 0.2 g/L of the fermentation medium. In some embodiments, the concentration of the L-Cysteine hydrochloride monohydrate in the fermentation medium is about 0.05 g/L, about 0.06 g/L, about 0.07 g/L, about 0.08 g/L, about 0.09 g/L, about 0.10 g/L, about 0.15 g/L, about 0.20 g/L, about 0.25 g/L, about 0.30 g/L, about 0.35 g/L, about 0.40 g/L, about 0.45 g/L, about 0.50 g/L, about 0.55 g/L, about 0.60 g/L, about 0.70 g/L, about 0.75 g/L, about 0.80 g/L, about 0.85 g/L, about 0.90 g/L, about 0.95 g/L, about 1.0 g/L, about 1.5 g/L, about 2.0 g/L, about 2.5 g/L, about 3.0 g/L, about 3.5 g/L, about 4.0 g/L, about 4.5 g/L, or about 5.0 g/L.

In accordance with the present disclosure, the fermentation medium may comprise medical antifoam c emulsion (Dow Corning®). In some embodiments, the concentration of medical antifoam c emulsion in the fermentation medium is between about 0.05 g/L and about 0.50 g/L, between about 0.10 g/L and about 0.40 g/L, between about 0.20 g/L and about 0.30 g/L, or between about 0.22 g/L and about 0.26 g/L (or ranges in between). In some embodiments, the concentration of medical antifoam c emulsion in the fermentation medium is about 0.05 g/L, about 0.10 g/L, about 0.12 g/L, about 0.14 g/L, about 0.16 g/L, about 0.18 g/L, about 0.20 g/L, about 0.22 g/L, about 0.24 g/L, about 0.26 g/L, about 0.28 g/L, about 0.30 g/L, about 0.32 g/L, about 0.34 g/L, about 0.36 g/L, about 0.38 g/L, about 0.40 g/L, about 0.45 g/L, or about 0.50 g/L, or any value in between. In an embodiment, the concentration of medical antifoam c emulsion in the fermentation medium is about 0.24 g/L.

In accordance with the present disclosure, the VTPM has a pH between 5 and 8, preferably between 6 and 7.8, for example about 6.1, 6.3, 6.5, 6.7, 6.9, 7.0, 7.1, 7.3, 7.5, and 7.7.

Culture Conditions

In accordance with the present disclosure, the first step of producing botulinum toxin is pre-cultivation of a Clostridium botulinum bacterium from a working cell bank (WCB). In a specific embodiment, the WCB is produced by first isolating a unique C. botulinum type A1 strain from a soil sample. The strain is further cultivated to generate spores and is frozen in multiple (e.g., 100) 0.5 mL aliquots as a master cell bank (MCB). An individual aliquot of the MCB is further cultivated to generate spores that are frozen in multiple (e.g., about 500) 0.5 mL aliquots as the WCB.

In a specific embodiment, WCB is thawed and added to a fermentation bag containing vegetable toxin producing media (VTPM). In a specific embodiment, the fermentation bag is a sterile, single-use, flexible fermentation bag containing ports and/or tubing for media inlet, inoculation, sample withdrawal, gas inlet, and gas outlet. In a specific embodiment, the fermentation bag includes a 0.2-μm gas filter on the gas inlet port and/or tubing to ensure a sterile environment. In a preferred embodiment, the VTPM is pre-heated to about 37±1° C. and flushed with filtered nitrogen gas to achieve anaerobic environment.

The anaerobic environment is defined as an environment with dissolved oxygen (DO)<2%. In some embodiments, the dissolved oxygen (DO) may be <2.0%, <1.9%, <1.8%, <1.7%, <1.6%, <1.5%, <1.4%, <1.3%, <1.2%, <1.1%, <1.0%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02%, or <0.01%. In a specific embodiment, the dissolved oxygen may be about 0%. In a specific embodiment, the WCB is thawed in room temperature for five minutes, then vortexed 3 times for 5 seconds each time before addition of 400 μl WCB to the fermentation bag containing 500 mL VTPM.

The fermentation process continues until OD₆₀₀ reaches an acceptable value, for example between about 0.1 and about 1.0, between about 0.1 and about 0.5, or preferably between about 0.2 and about 0.4 (or ranges in between) to produce a pre-culture. In some embodiments, the OD₆₀₀ reaches a value of about 0.1, about 0.2, about 0.3, about 0.4, about 0.5, about 0.6, about 0.7, about 0.8, about 0.9, or about 1.0.

In a specific embodiment, a test of microbiological purity for other microorganisms than C. botulinum, is performed as pre-culture in process control. In a specific embodiment, the test is performed to detect possible contamination of the bacterial culture during fermentation. Only Clostridium botulinum should be present and detected. To screen for the possible presence of any contaminating bacteria or fungi in the C. botulinum culture, the test is performed in different media and environmental conditions. In a specific embodiment, to detect anaerobic bacteria, 10 μL of the culture is streaked on a sheep blood agar plate which is incubated at 30-35° C. under anaerobic conditions. In a specific embodiment, to detect aerobic bacteria, 1 mL of the culture is mixed with TSA and incubated at 30-35° C. In a specific embodiment, to detect yeast and molds, a sample of 1 mL is mixed with SAB and incubated at 20-25° C. There should be no growth on the TSA and SAB plates, and all colonies growing on the sheep blood plate should have the same (Clostridial) morphology. The subsequent Gram-staining of colonies from the blood plate should show Grampositive rod-shaped bacteria and spores. Viable count of C. botulinum is analyzed for in-process monitoring.

The next step of producing botulinum toxin is main culture. In a specific embodiment, the pre-culture is added to a fermentation bag containing 4500 ml VTPM, pre-heated to 33±1° C. and flushed with filtered nitrogen gas to achieve anaerobic environment. The anaerobic environment is defined as an environment with dissolved oxygen (DO) <2%. In some embodiments, the dissolved oxygen (DO) may be <1.9%, <1.8%, <1.7%, <1.6%, <1.5%, <1.4%, <1.3%, <1.2%, <1.1%, <1.0%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02%, or <0.01% (or ranges in between). In a specific embodiment, the dissolved oxygen may be about 0%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, or about 2.0%.

In some embodiments, the fermentation process continues for between about 60 hours and about 80 hours, between about 65 hours and about 75 hours, or between about 67 hours and about 71 hours (or ranges in between). In some embodiments, the fermentation process continues for about 60 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, or about 80 hours. In some embodiments, the fermentation process continues for about 69±2 hours, about 69±1 hours, about 69±0.5 hours, or about 69±0.2 hours. In an embodiment, the step (c) is conducted for about 69 hours.

In a specific embodiment, a test of microbiological purity for other microorganisms than C. botulinum, is performed as pre-culture in process control. Viable count of C. botulinum is analyzed for in-process monitoring.

### EXAMPLES

### Example 1

### Pre-Cultivation

To a 2 L fermentation bag, 500 mL of Vegetable Toxin Producing Media (VTPM) (Table 1), was added, pre-heated to 37° C. and flushed with filtered nitrogen gas to achieve anaerobic environment. In-process control of dissolved oxygen (DO) was performed until the DO was <2%. One vial with working cell bank (WCB) was thawed at room temperature for five minutes, then vortexed 3 times for 5 seconds each time before addition of 400 μl WCB using pipette under grade A air supply to the fermentation bag that was then placed on the bioreactor.

For the working cell bank (WCB), a proprietary cell bank was used. Briefly, botulinum toxin type A1 is isolated from a soil sample. For this WCB, the toxin operon is 100% identical to Strain Hall, ATCC 3502, a representative of the Group I (proteolytic) botulinum toxin producing bacteria.

The temperature was set to 37±1° C., the agitation angle to 12° and the oscillation frequency to 12 min⁻¹. The oxygen level (DO) and pH was monitored in real-time. The fermentation continued and in-process control of OD₆₀₀ was taken until the OD₆₀₀ has reached a value in the range of 0.2 to 0.4, after approximately 19 hours.

At the end of the pre cultivation step, a test of microbiological purity, for other microorganisms than C. botulinum, was performed as pre-culture In-process control. Viable count of C. botulinum was analyzed for in-process monitoring.

TABLE 1
Composition of Vegetable Toxin Production Medium (VTPM)

| Raw Material | Amount (per L) | |
|---|---|---|
| 1. Wheat peptone (Solabia, A2101) | 20.0 ± 0.2 | g |
| 2. Yeast extract (BD Biosciences, 212750) | 20.0 ± 0.2 | g |
| 3. D-(+)-Glucose | 5.0 ± 0.05 | g |
| 4. L-Cysteine hydrochloride monohydrate | 0.20 ± 0.02 | g |
| 5. Medical Antifoam C emulsion | 0.25 ± 0.025 | g |
| 6. Distilled water | 970 ± 10 | mL |

| | |
|---|---|
| 7. NaOH | for pH-adjustments |
| 8. HCl | for pH-adjustments |

### Example 2

### Main Culture and Fermentation

A 10 L fermentation bag was filled with 4500 ml of VTPM under nitrogen gas flow, pre-heated to 33±1° C. on a bioreactor with an agitation angle of 12° and an oscillation frequency of 12 min⁻¹. The pre-culture from the previous step was added by siphoning to the fermentation bag. The bag was flushed with nitrogen gas to achieve anaerobic environment and in-process control of dissolved oxygen (DO) was performed until DO stabilized at <2%.

The fermentation was continued for 69±2 h from point of main culture inoculation. In-process control of harvest culture is performed by testing microbiological purity with respect to microorganisms other than C. botulinum. Viable count of C. botulinum is analyzed for in-process monitoring.

Briefly, the test for microbiological purity is performed to detect possible contamination of the bacterial culture during fermentation. Only Clostridium botulinum should be present and detected. To screen for the possible presence of any contaminating bacteria or fungi in the C. botulinum culture, the test is performed in different media and environmental conditions. To detect anaerobic bacteria, 10 μL of the culture is streaked on a sheep blood agar plate which is incubated at 30-35° C. under anaerobic conditions. To detect aerobic bacteria, 1 mL of the culture is mixed with TSA and incubated at 30-35° C. To detect yeast and molds, 1 mL of the culture is mixed with SAB and incubated at 20-25° C. There should be no growth on the TSA and SAB plates, and all colonies growing on the sheep blood plate should have the same (Clostridial) morphology. The subsequent Gram-staining of colonies from the blood plate should show Gram-positive rod-shaped bacteria and spores.

TABLE 2
Process Parameters and Ranges

| | |
|---|---|
| Parameter | Preferred range |

Pre-Cultivation

| VTPM | 500 ± 10 | g |
|---|---|---|
| Temperature | 37 ± 1° | C. |
| Inoculation volume WCB | 400 ± 7.5 | μl |
| Dissolved Oxygen (DO) at start | <2%, pref. <0.2% | |
| Fermentation time | 19 ± 3 | h |
| OD₆₀₀ | 0.2-0.4 | AU |
| Agitation angle | 12° | |
| Agitation speed | 12 | rpm |

Main Cultivation

| Inoculation volume from pre | 500 | ml |
|---|---|---|
| Cultivation | | |
| VTPM growth media | 4500 ± 10 | g |
| Temperature | 33 ± 1° | C. |
| DO at start | <2%, preferably <0.2% | |
| Fermentation time | 69 ± 2 | h |
| Agitation angle | 12° | |
| Agitation speed | 12 | rpm |

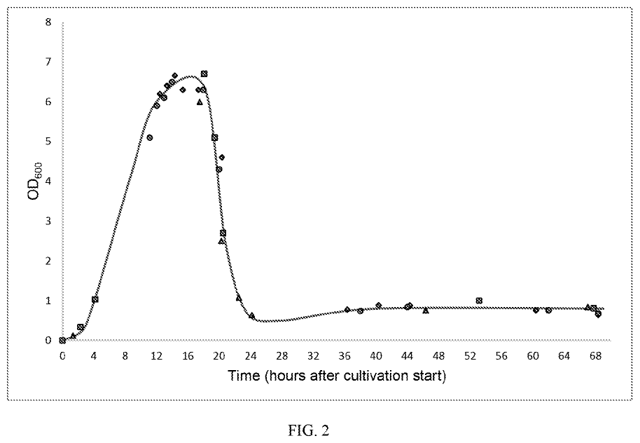

Growth of C. botulinum is monitored by sampling the main culture and measuring the optical density at 600 nm (OD₆₀₀). FIG. 2 shows an optical density curve (absorbance at 600 nm) for main cultivations of C. botulinum in VTPM carried out according to Example 2. The absorbance at 600 nm over time provides a growth curve for the main culture. Rapid growth is observed during the first 15 hours, where OD₆₀₀ increases up to around 7, after which there is an equally rapid decrease in OD₆₀₀ down to around 1, due to lysis of the bacteria and release of the toxin molecule. For the remaining time, from around 40 hours until harvest at 69±2 hours, the OD₆₀₀ is fairly stable, showing only a small increase.

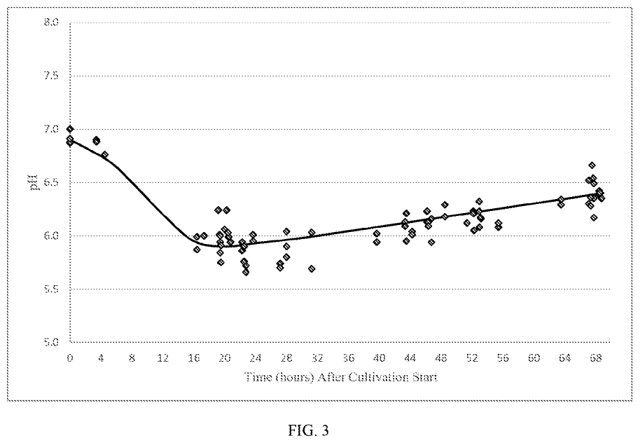

FIG. 3 shows results from in-line pH monitoring during fermentation of the main culture. The pH trace of the main culture exhibits a typical pattern with an initial drop from pH 7 down to around pH 5.7 at around 15 hours, which is approximately the same time at which OD₆₀₀ reaches its peak. After 15 hours, there is a slow but continuous increase of pH up to 6.3 at 69 hours (i.e., at harvest).

The toxin yield in the main culture at harvest can be measured with a BONT/A-specific ELISA. The average concentration generated in main cultures grown according to Example 2 is 4.9 μg/mL with a standard deviation of 0.75.

The ELISA protocol is an indirect sandwich ELISA based on the principles and general method described in USP <1103>, “Immunological Test Methods—Enzyme-linked Immunosorbent Assay.” The ELISA method is based on immunological binding and detection of BoNT/A using two different types of BoNT/A-specific polyclonal antibodies.

A series of protein standard dilutions based on a commercial BoNT/A toxin, is prepared by diluting BoNT/A in PBS-Tween solution (0.05% Tween-20), to the concentration range of 3-28 ng/mL. A sample diluted in PBS-Tween to the range of the protein standard dilutions, is added in triplicate to microplate wells coated with polyclonal anti-BoNT/A antibody. Incubation results in antibody recognition and binding of BoNT/A antigen to the well. Each incubation is followed by an automated washing step using PBS-Tween solution.

Primary detection is carried out by binding another type of polyclonal anti-BoNT/A antibody, leading to formation of the sandwich complex. A secondary antibody conjugated to horseradish peroxidase (HRP) is then added. Its binding to the primary antibody allows for detection of BoNT/A within the sandwich complex. 3,3′,5,5′-tetramethylbenzidine (TMB) substrate is then added to the sample wells. HRP converts TMB substrate to produce a blue reaction product. A stop solution is added, halting the TMB conversion and initiating a color conversion of remaining TMB to yellow. Absorbance in each microplate well is detected at 450 nm with a plate reader, with the measured absorbance being directly proportional to the amount of BoNT/A in the well. The sample absorbance values are calculated by comparison against a standard curve based on absorbance values from the BoNT/A standard dilutions. The results are reported as mean values, μg/mL.

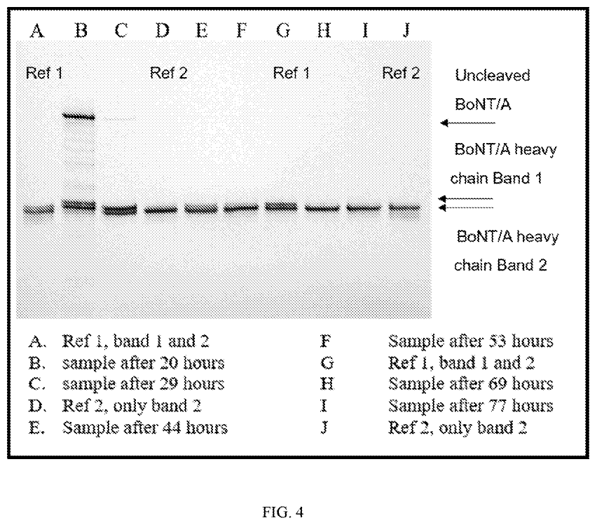

During the fermentation of the main culture, toxin becomes detectable in the culturing media after around 15 hours. When the toxin content in reduced fermentation samples of the main culture are analyzed by Western blot analysis with a polyclonal anti-BoNT/A antibody, different toxin heavy chain variants are detected. FIG. 4 shows an example of such a Western blot analysis, which tracks heavy chain band 1 and 2 formation during main fermentation (samples from 20-77 hours).

During the early phases of the fermentation, there are three visible main bands, representing uncleaved pre-toxin polypeptide of 160 kDa, band 1 of the heavy chain at around 100 kDa, and band 2 of the heavy chain running just below band 1 of the fully mature BoNT/A. During the fermentation, the uncleaved pre-toxin polypeptide and heavy chain band 1 gradually disappear and are converted into the mature heavy chain band 2 isoform. At harvest (69±2 hours), only the mature heavy chain band 2 isoform is present in the main culture.

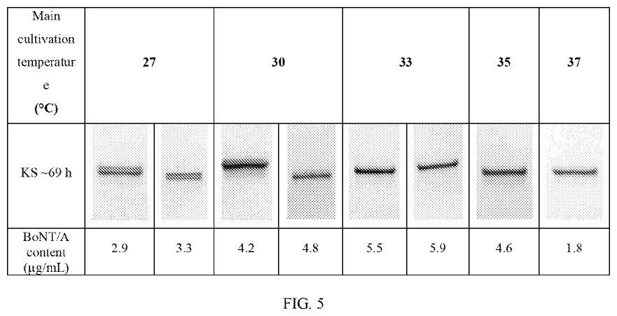

Maturation of the BoNT/A protein into the band 2 heavy chain isoform is controlled by the fermentation time, but the fermentation temperature is another important factor. FIG. 5 shows a table with cut-out figures (from Western blot analysis) of BoNT/A heavy chain variants at harvest of main cultivations carried out at different temperatures. The table also provides BoNT/A concentration for the same samples (as determined by ELISA). For temperatures at or below 30° C., the maturation is not fully complete at 69 hours of fermentation of the main culture. At a fermentation temperature at or above 35° C., the maturation into the band 2 heavy chain isoform is complete at 69 hours, but the concentration of BoNT/A in the culturing media is lower. Taken together, the data reveal that the optimal temperature for the main cultivation is about 33° C., which permits generating a fully mature BoNT/A, with a high yield of the toxin.

### Example 3

### Comparison with Other Plant Derived Peptones

Apart from wheat peptone, the VTPM media can be based on other plant peptones (e.g., soybean, potato or broadbean peptones) for the growth of C. botulinum and the production of botulinum toxin.

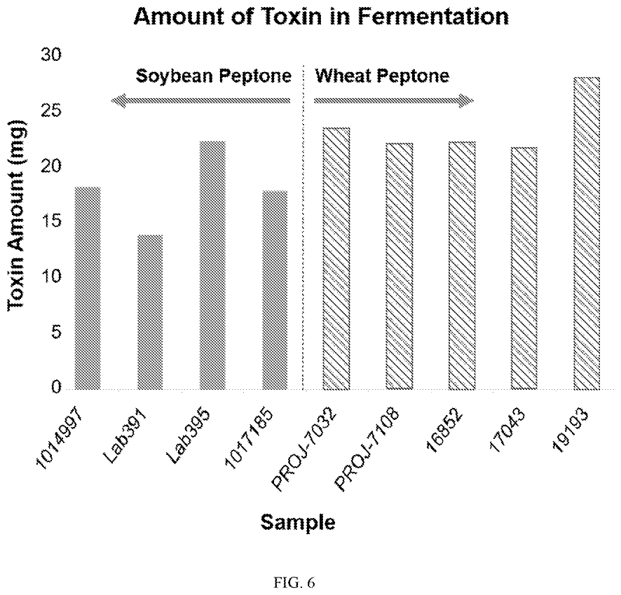

FIG. 6 shows amount of botulinum toxin obtained in main cultures of C. botulinum grown at 30° C. in a VTPM based on soybean peptone (solid bars) or a VTPM based on wheat peptone (patterned bars), of equal total volumes. The data show that wheat peptone gives a somewhat higher toxin yield but also a more robust, consistent process.

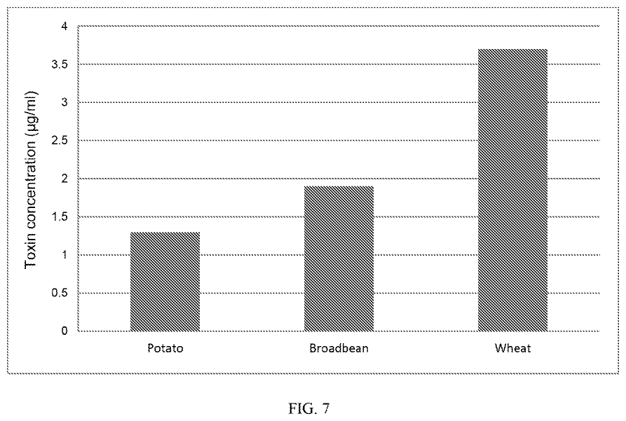

FIG. 7 shows toxin concentration obtained in main cultures of C. botulinum grown at 30° C. in VTPM based on potato, broadbean, or wheat peptone. All three VTPM media give toxin concentrations at harvest that are above 1 μg/mL. However, wheat peptone yields a substantially higher amount of toxin (approximately 4 μg/mL) than both potato and broadbean peptone-based VTPMs.

What is claimed is:

1. A method for production of a botulinum toxin, comprising the steps of:
(a) providing a working cell bank (WBC) comprising a Clostridum botulinum bacterium;
(b) adding the working cell bank to a first container containing a vegetable toxin production medium (VTPM), and culturing the Clostridium botulinum bacterium in the VTPM under conditions which permit growth of the Clostridium botulinum to produce a pre-culture;
(c) adding the pre-culture to a second container containing VTPM, and culturing the Clostridium botulinum bacterium under conditions that allow production of a botulinum toxin, and;
(d) recovering the botulinum toxin;
wherein the VTPM is substantially free or free of an animal derived product and comprises at least one plant-derived protein.

2. The method of claim 1, wherein the botulinum toxin is botulinum neurotoxin type A (BoNT/A).

3. The method of claim 1 or 2, wherein the container used in the steps (b) and (c) is a fermentation bag.

4. The method of any one of claims 1-3, wherein the conditions in steps (b) and (c) comprise an anaerobic environment.

5. The method of claim 4, wherein the anaerobic environment has a dissolved oxygen (DO) concentration of <2%.

6. The method of any one of claims 1-5, wherein the condition in step (b) comprises a temperature of between about 35° C. and about 39° C., preferably about 37±1° C.

7. The method of any one of claims 1-6, wherein the condition in step (c) comprises a temperature of between about 30° C. and about 37° C., preferably about 33±1° C.

8. The method of any one of claims 1-7, wherein the volume ratio of the WCB to the VTPM in step (b) is no greater than about 2.0%, preferably about 0.08%.

9. The method of any one of claims 1-8, wherein the volume ratio of the pre-culture to the VTPM in step (c) is between about 1:2 and about 1:50, preferably about 1:9.

10. The method of any one of claims 1-9, wherein the step (b) is conducted until OD₆₀₀ reaches the range of about 0.1 to about 1.0, preferably about 0.2 to about 0.4.

11. The method of any one of claims 1-10, wherein the step (b) is conducted for between about 10 and about 30 hours, preferably about 19 hours.

12. The method of any one of claims 1-11, wherein the step (c) is conducted for between about 60 and about 80 hours, preferably for about 69±2 hours.

13. The method of any one of claims 1-12, wherein microbiological purity for other microorganisms than C. botulinum is tested in the pre-culture after step (b) and before step (c).

14. The method of any one of claims 1-13, wherein microbiological purity for other microorganisms than C. botulinum is tested in the culture after step (c) and before step (d).

15. The method of any one of claims 1-14, wherein the plant-derived protein is a wheat peptone, broadbean peptone, potato peptone, pea peptone, rice peptone, or soybean peptone, preferably a wheat peptone.

16. The method of any one of claims 1-15, wherein the wheat peptone concentration in the VTPM is between about 10 g/L and about 30 g/L, preferably about 20 g/L.

17. The method of any one of claims 1-16, wherein the VTPM comprises wheat peptone, yeast extract, D-(+)-Glucose, L-Cysteine hydrochloride monohydrate, and Medical antifoam C emulsion.

18. The method of any one of claims 1-17, wherein the VTPM comprises:
between about 5 g/L and about 50 g/L, preferably about 20 g/L, of wheat peptone;
between about 5 g/L and about 50 g/L, preferably about 20 g/L, of yeast extract;
between about 0.05 g/L and about 20 g/L, preferably about 5 g/L, of D-(+)-glucose;
between about 0.05 g/L and about 0.50 g/L, preferably about 0.20 g/L, of L-cysteine hydrochloride monohydrate; and
between about 0.05 g/L and about 0.50 g/L, preferably about 0.24 g/L, of medical antifoam c emulsion.

19. The method of any one of claims 1-18, wherein the pH of the VPTM is between about 6.7 and about 7.2.

20. A composition comprising a Clostridium botulinum and a culture medium for producing a botulinum toxin, wherein the medium is free or substantially free of an animal derived product, and comprises at least one plant-derived protein.

21. The composition of claim 20, wherein the plant-derived protein is a wheat peptone.

22. The composition of claim 21, wherein the wheat peptone concentration in the VTPM is between about 5 g/L and about 50 g/L, preferably about 20 g/L.

23. The composition of any one of claims 20-22, wherein the culture medium comprises wheat peptone, yeast extract, D-(+)-Glucose, L-Cysteine hydrochloride monohydrate, and Medical antifoam C emulsion.

24. The composition of any one of claims 20-23, wherein the culture medium comprises:
between about 5 g/L and about 50 g/L, preferably about 20 g/L, of wheat peptone;
between about 5 g/L and about 50 g/L, preferably about 20 g/L, of yeast extract;
between about 1 g/L and about 20 g/L, preferably about 5 g/L, of D-(+)-glucose;
between about 0.05 g/L and about 0.50 g/L, preferably about 0.20 g/L, of L-cysteine hydrochloride monohydrate; and
between about 0.05 g/L and about 0.50 g/L, preferably about 0.24 g/L, of medical antifoam c emulsion.
